(19) United States
(12) Patent Application Publication
(10) Pub. No.: US 2021/0007625 A1
(43) Pub. Date: Jan. 14, 2021

(54) ELECTRODE AND SENSOR

(71) Applicant: SONY CORPORATION, TOKYO (JP)

(72) Inventor: MAO KATSUHARA, KANAGAWA (JP)

(21) Appl. No.: 16/968,686

(22) PCT Filed: Feb. 5, 2019
(86) PCT No.: PCT/JP2019/003962

§ 371 Date: Aug. 10, 2020

(30) Foreign Application Priority Data
Feb. 16, 2018 (JP) 2018-026342

(51) Int. Cl. A61B 5/053

(52) U.S. Cl. CPC A61B 5/053

(57) ABSTRACT

An electrode according to an embodiment of the present disclosure includes: a first conductive material; a second conductive material having non-polarizable property and ionic bonding; and a substrate that includes the first conductive material and the second conductive material, and has a first region and a second region that are different from each other in a concentration ratio between the first conductive material and the second conductive material.

### TECHNICAL FIELD

The present disclosure relates to an electrode that is used in, for example, a biopotential measurement, and a sensor that includes such an electrode.

### BACKGROUND ART

Typically, a biopotential measurement uses a wet electrode. The measurement with use of the wet electrode involves applying an electrolyte gel between the electrode and skin, which raises an issue with deterioration in characteristics over time due to evaporation of the moisture contained in the electrolyte gel, or contamination caused by the electrolyte gel.

Therefore, a dry electrode has been proposed that avoids the use of the electrolyte gel. In recent years, to form a biopotential electrode with superior mountability, a method has been proposed that forms an electrode resin by mixing conductive particles such as, for example, carbon in an elastomer (for example, see NPTL 1). Further, an electrode has been proposed that includes carbon-mixed resin and has a silver-chloride (AgCl)-coated portion at a contact portion to be brought into contact with a living body (for example, see NPTL 2).

### CITATION LIST

### Non-Patent Literature

NPTL 1: Sensors, 2014, 14, 23758-23780

NPTL 1: Sensors, 2014, 14, 12847-12870

### SUMMARY OF THE INVENTION

Incidentally, an electrode for a biopotential measurement is required to improve mechanical and electrical reliability.

It is therefore desirable to provide an electrode and a sensor that make it possible to improve the mechanical and electrical reliability.

An electrode according to an embodiment of the present disclosure includes: a first conductive material; a second conductive material having non-polarizable property and ionic bonding; and a substrate that includes the first conductive material and the second conductive material, and has a first region and a second region that are different from each other in a concentration ratio between the first conductive material and the second conductive material.

A sensor according to an embodiment of the present disclosure includes the above-described electrode according to the embodiment of the present disclosure as a measurement section that measures information on an object.

In the electrode according to the embodiment and the sensor according to the embodiment of the present disclosure, the first region and the second region are formed, in the substrate, that are different from each other in the concentration ratio between the first conductive material and the second conductive material having the non-polarizable property and the ionic bonding. The use of the region with the higher concentration ratio of the second conductive material having the non-polarizable property and the ionic bonding between two kinds of the above-described conductive materials as a contact portion to be brought into contact with an object (a living body) prevents polarization of the contact portion brought into contact with the living body. Further, the regions that are different from each other in the concentration ratio between the first conductive material and the second conductive material are formed in an integrated manner, which reduces a possibility that the region containing the second conductive material in higher concentration will drop off.

According to the electrode of the embodiment and the sensor of the embodiment of the present disclosure, the regions are provided that are different from each other in the concentration ratio in the substrate that includes the first conductive material and the second conductive material having the non-polarizable property and the ionic bonding. Therefore, the use of the region containing the above-described second conductive material in higher concentration as the contact portion to be brought into contact with the living body reduces generation of polarization caused by contact with the living body. This makes it possible to measure a potential accurately, allowing for improvement in the electrical reliability. Further, the regions that are different from each other in the concentration ratio between the first conductive material and the second conductive material are formed in an integrated manner, allowing for improvement in the mechanical reliability.

It is to be noted that effects described above are not necessarily limitative, and any of effects described in the present disclosure may be provided.

### BRIEF DESCRIPTION OF DRAWINGS

FIG. 1 is a planar schematic view (A) and a schematic cross-sectional view (B) that illustrate an example of a configuration of an electrode according to an embodiment of the present disclosure.

FIG. 2A is a schematic cross-sectional view of another example of the configuration of the electrode illustrated in FIG. 1.

FIG. 2B is a schematic cross-sectional view of another example of the configuration of the electrode illustrated in FIG. 1.

FIG. 3A is a schematic planar view of another example of the configuration of the electrode according to the embodiment of the present disclosure.

FIG. 3B is a schematic planar view of another example of the configuration of the electrode according to the embodiment of the present disclosure.

FIG. 3C is a schematic planar view of another example of the configuration of the electrode according to the embodiment of the present disclosure.

FIG. 4A is a schematic diagram illustrating an example of a method of manufacturing the electrode illustrated in FIG. 3C.

FIG. 4B is a schematic diagram illustrating a process subsequent to the process illustrated in FIG. 4A.

FIG. 4C is a schematic diagram illustrating a process subsequent to the process illustrated in FIG. 4B.

FIG. 5A is a schematic diagram illustrating another example of the method of manufacturing the electrode illustrated in FIG. 3C.

FIG. 5B is a schematic diagram illustrating a process subsequent to the process illustrated in FIG. 5A.

FIG. 5C is a schematic diagram illustrating a process subsequent to the process illustrated in FIG. 5B.

FIG. 6 is a diagram illustrating an application example 1.

FIG. 7 is a diagram illustrating an application example 2.

### MODES FOR CARRYING OUT THE INVENTION

Hereinafter, embodiments of the present disclosure are described in detail with reference to the drawings. The following description is merely a specific example of the present disclosure, and the present disclosure is not limited to the following embodiments. Further, the present disclosure is not limited to arrangements, dimensions, dimension ratios, and the like of the components illustrated in the drawings. It is to be noted that descriptions are given in the following order.
1. Embodiment (an example where regions having different concentration ratios between two kinds of contained conductive materials are provided in an electrode)

1-1. Configuration of Electrode

1-2. Method of Manufacturing Electrode

1-3. Workings and Effects
2. Application Examples

### 1. EMBODIMENT

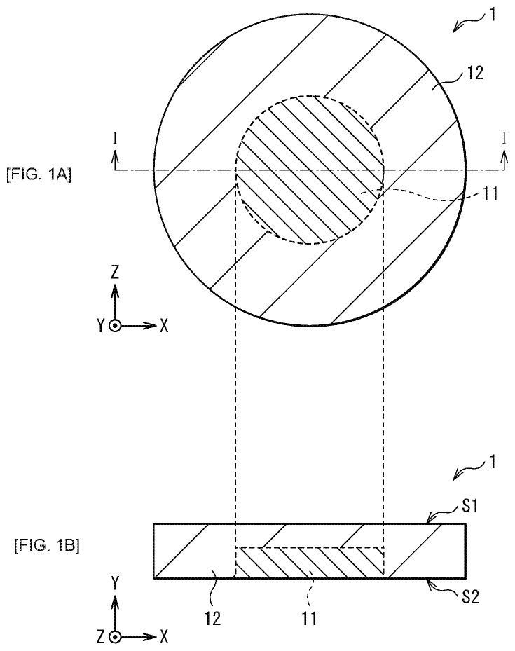

Part (A) of FIG. 1 schematically illustrates an example of a planar configuration of an electrode (an electrode 1) according to an embodiment of the present disclosure, and Part (B) of FIG. 1 schematically illustrates an example of a cross-sectional configuration of the electrode 1 taken along a line I-I illustrated in Part (A) of FIG. 1. The electrode 1 is used as an electrode of a biological sensor that is brought into contact with a living body to measure the potential, for example. The electrode 1 of the present embodiment has a configuration in which, in a substrate forming the electrode 1, a conductive material (a first conductive material) and a conductive material having non-polarizable property and ionic bonding (a second conductive material, hereinafter referred to as a non-polarizable material) are included, and a first region 11 and a second region 12 are formed that are different from each other in the concentration ratio between the conductive material and the non-polarizable material. It is to be noted that FIG. 1 schematically illustrates an example of a configuration of the electrode 1 with dimensions and shapes that may be different from actual ones.

### (1-1. Configuration of Electrode)

The substrate is a base material that forms the electrode 1, and in which the above-described conductive material and non-polarizable material are dispersed. Specific examples of the material include thermoplastic resin, such as polyvinyl chloride (PVC), polypropylene (PP), polyethylene (PE), polyurethane (PU), polyacetal (POM), polyamide (PA), and polycarbonate (PC), or a copolymer of any of these materials. Besides the above, a thermosetting elastomer, such as silicon resin and polyurethane resin may be used. As an alternative, diene-based rubber, such as natural rubber, styrene butadiene rubber, or isoprene rubber may be used.

The conductive material has higher electrical conductivity than the non-polarizable material in the substrate, and is, for example, particles that contain carbon as a main constituent. Here, the main constituent is defined as a constituent having the highest composition ratio (volume/specific gravity) among constituents contained in the substrate. Specific examples of the material include graphite-based particles such as carbon black and Ketchen black; carbon-based particles such as fullerene and carbon nanotube; carbon-based material particles such as graphene particles; and metal particles such as gold, silver, or copper, or a nanowire of any of these materials.

The non-polarizable material is a conductive material having the non-polarizable property and the ionic bonding, as described above. Specific examples of the material include a metal compound such as silver chloride (AgCl) or copper sulfide (CuS); a metal oxide such as palladium oxide (PdO₂) or indium tin oxide (ITO); and a conductive polymer such as PEDOT-PSS, PEDOT-TsO, or polyaniline in the form of particles or fibers.

As described above, in the substrate of the electrode 1 of the present embodiment, the conductive material and the non-polarizable material are dispersed, and the first region 11 and the second region 12 are formed that are different from each other in the concentration ratio. For example, as illustrated in Part (A) of FIG. 1, the electrode 1 is a circular electrode having a certain thickness in a Z-axis direction, for example, and paired opposite surfaces (a front surface (a surface S1) and a rear surface (a surface S2)). For example, the first region 11 is provided in a central part on the surface S2 side, and the second region 12 is provided to cover the first region 11. It is to be noted that the surface S2 side of the electrode 1 is a contact surface to be brought into contact with an object, and the first region 11 is provided on the contact surface side.

The first region 11 is a region in which the non-polarizable material is dispersed in a higher concentration than that of the conductive material. The second region 12 is a region in which the conductive material is higher in concentration, and the non-polarizable material is lower in concentration, as compared with the first region 11. The first region 11 is, for example, a contact portion to be brought into contact with an object when the electrode 1 is mounted. In this way, in a case where the object is a living body, for example, the non-polarizable material dispersed in high concentration in the contact portion to be brought into contact with the object prevents polarization caused by contact with the living body, which allows for an accurate biopotential measurement.

It is to be noted that, in the vicinity of an interface between the first region 11 and the second region 12, a concentration gradient where the conductive material and the non-polarizable material vary continuously in concentration may be formed.

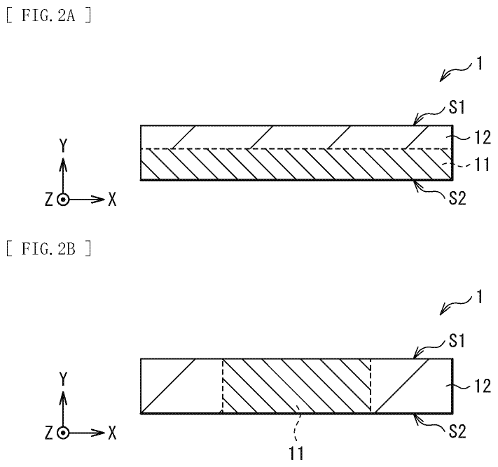

Further, FIG. 1 illustrates an example of providing the first region 11 at the center of electrode 1 on the surface S2 side to be brought into contact with an object; however, a method of providing the first region 11 is not limited thereto. For example, as illustrated in FIG. 2A, the first region 11 may be provided on a whole surface of the surface S2 side. Alternatively, an upper side does not necessarily have to be covered with the second region 12, as illustrated in FIG. 1 (B). For example, as illustrated in FIG. 2B, the first region 11 may be provided at the center of the electrode 1, and the second region 12 may be formed only around the first region 11.

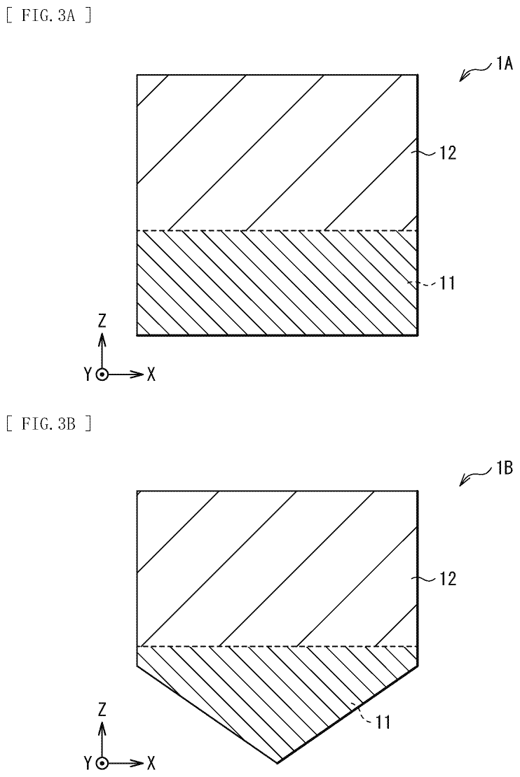

Further, a planar shape of the electrode 1 is not limited to the circular shape as illustrated in FIG. 1(A). For example, the shape may be a rectangular form like an electrode 1A illustrated in FIG. 3A, or a pentagonal form like an electrode 1B illustrated in FIG. 3B. In addition, for example, in a case where the electrode 1 is mounted on a body portion having body hair, such as the head, the shape may be a comb form like an electrode 1C illustrated in FIG. 3C, for example. In a case where the comb form is to be used, the first region 11 is preferably formed on tip portions of comb teeth, as illustrated in FIG. 3C. When such an electrode 1C is put in hair, the first region 11 formed on the tip portions of the comb teeth is brought into contact with the skin through gaps in the hair, which allows for good contact with the skin having body hair.

### (1-2. Method of Manufacturing Electrode)

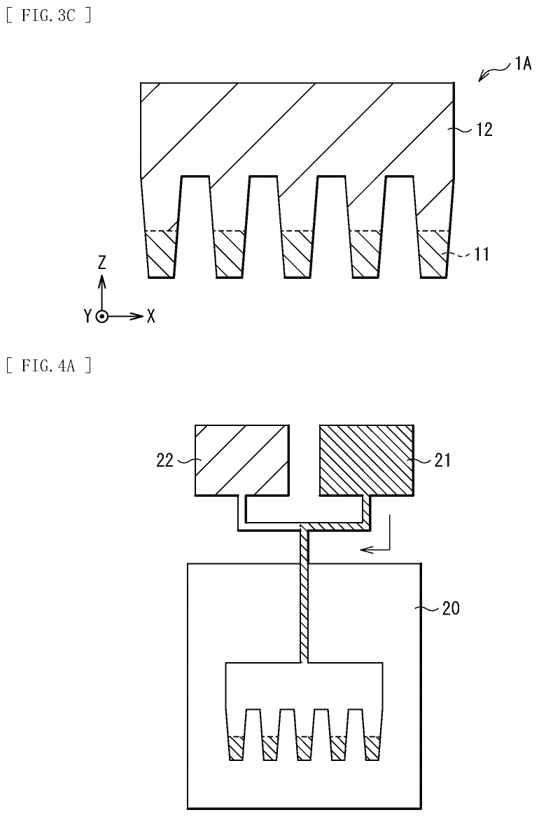

Descriptions are provided of a method of manufacturing the electrode 1 of the present embodiment. It is to be noted that manufacturing processes of the comb-shaped electrode 1C illustrated in FIG. 3C are here described with reference to FIG. 4A to FIG. 4C.

First, a polyurethane resin elastomer, for example, is used as the substrate, and, Ketchen black, for example, is kneaded into the elastomer at a rate of, for example, six wt % as the conductive material. Further, the polyurethane resin elastomer, for example, is used as the substrate, and, silver chloride (AgCl), for example, is kneaded into the elastomer at a rate of, for example, 20 wt % as the non-polarizable material. Next, the polyurethane resin elastomer into which the Ketchen black is kneaded is set into an extrusion molding machine 22, and the polyurethane resin elastomer into which the silver chloride (AgCl) is kneaded is set into an extrusion molding machine 21.

Next, injection molding is performed while changing the injection amount (rate) of the extrusion molding machines 21 and 22 in accordance with a shape of the electrode 1. For example, in the comb-shaped electrode C as illustrated in FIG. 3C, 100% of the silver chloride (AgCl)-containing polyurethane resin elastomer is first injected into the tip portions of the comb teeth section from the extrusion molding machine 21, as illustrated in FIG. 4A. Subsequently, as illustrated in FIG. 4B, the silver chloride (AgCl)-containing polyurethane resin elastomer and the Ketchen black-containing polyurethane resin elastomer are injected respectively from the extrusion molding machines 21 and 22 while changing the injection amounts.

Thereafter, as illustrated in FIG. 4C, 100% of the Ketchen black-containing polyurethane resin elastomer is injected from the extrusion molding machine 22 to form the comb-shaped electrode 1C. Finally, a molding die 20 is cooled to take out the electrode 1C. Steps as described above bring the electrode 1C illustrated in FIG. 3C to completion.

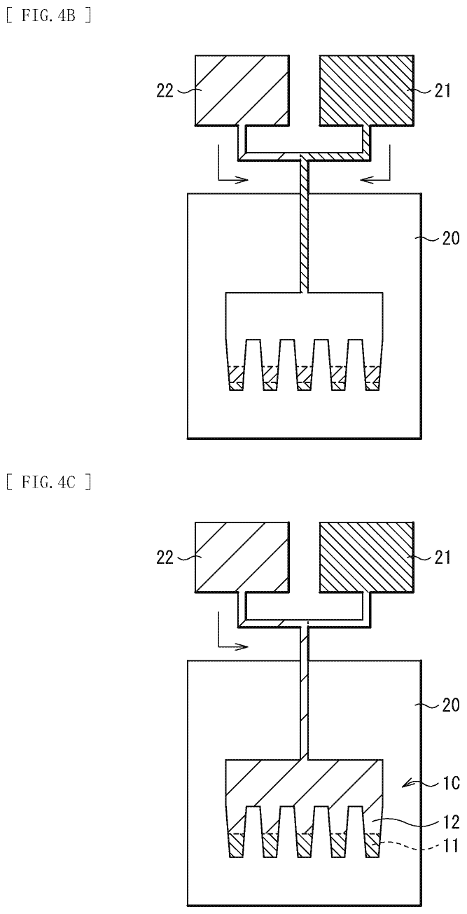

Further, it is possible to manufacture the electrode 1C of the present embodiment in a more simplified manner by using a method illustrated in FIG. 5A to FIG. 5C.

First, thermosetting silicon resin, for example, is used as the substrate, and Ketchen black with a particle size of, for example, about 40 nm to be used as the conductive material, and silver chloride (AgCl) with a particle size of, for example, about 1 μm to be used as the non-polarizable material are mixed in the silicon resin using a stirring machine at a rate of six wt % of Ketchen black and 10 wt % of AgCl, respectively. Subsequently, as illustrated in FIG. 5A, mixed resin 13 that is a mixture of the Ketchen black and the silver chloride (AgCl) is injected into the molding die 20.

Next, as illustrated in FIG. 5B, the molding die 20 is set into a centrifugal machine to be rotated prior to curing of the resin, for example. This removes any bubbles contained in the substrate and causes the non-polarizable material to locally aggregate at the tip portions of the comb teeth.

Subsequently, the molding die 20 is heated to cure the mixed resin 13 and then cooled to take out the electrode 1C. Steps as described above bring the electrode 1C illustrated in FIG. 3C to completion.

In a case where the electrode 1 (1C) having the first region 11 and the second region 12 that are different from each other in the concentration ratio between the conductive material and the non-polarizable material is manufactured using the above-described method, it is preferable to add a difference in dispersibility between the conductive material and the non-polarizable material relative to the substrate. Specifically, it is preferable to make the dispersibility of the conductive material greater than the dispersibility of the non-polarizable material.

An example of a method of increasing the dispersibility of the conductive material includes a method of making an average primary particle size of the conductive material smaller than an average primary particle size of the non-polarizable material. Further, the example also includes a method of making a specific gravity of the conductive material smaller than a specific gravity of the non-polarizable material. In specific, for example, a method is provided that introduces a polycarboxylic acid-based, urethane-based, or acrylic resin-based modifying group on a surface of the conductive material. Examples of a coupling agent that binds such a modifying group with a particle include a triisostearoyl titanate-based coupling agent, a silane coupling agent, thiol, or phosphate ester. After the silicon resin that is the mixture of the Ketchen black and the silver chloride (AgCl) is injected into the molding die 20 while the dispersibility of the conductive material and the non-polarizable material is adjusted, the molding die 20 is held while being kept at temperature of a softening point of the resin or higher. This makes it possible to form a concentration distribution of the Ketchen black and the silver chloride (AgCl).

It is to be noted that, in the above-described methods, an example is cited in which one kind of conductive material and one kind of non-polarizable material are dispersed in the substrate; however, the example is non-limitative. Two or more kinds of materials may be used for each of the conductive material and the non-polarizable material. Further, the above-described manufacturing method is merely one example, and any other method may be used for manufacturing.

### (1-3. Workings and Effects)

As described above, a wet electrode is typically used for a biopotential measurement. The wet electrode makes it possible to reduce a contact impedance of a living body by interposing an electrolyte gel between a metal electrode and skin. However, a measurement with use of the wet electrode using the electrolyte gel raises an issue with deterioration in characteristics over time due to evaporation of the moisture contained in the electrolyte gel, or contamination caused by the electrolyte gel.

Therefore, a dry electrode has been proposed that avoids interposition of the electrolyte gel. A representative example of the dry electrode includes metal or a metal compound. An issue with the dry electrode includes difficulty in getting good contact with the skin of a body portion having body hair, such as the head, and difficulty with accurate potential measurement. As a method of solving such an issue, a typical method is adopted that uses a comb-shaped electrode to contact with the skin through gaps in the hair; however, pain or difficulty in mounting still remains as an issue. Further, in a portion with less body hair, it is pointed out that degradation in the signal quality is caused because a state of good contact with the skin is unobtainable due to hardness of metal, or deterioration in the mountability is caused due to incrustation or the like.

In recent years, as a method of forming a biopotential electrode that is superior in terms of mountability, a method has been proposed that forms an electrode resin by mixing conductive particles in an elastomer. Such a method typically uses carbon or the like as the conductive particle; however, carbon-mixed resin polarizes due to contact with a living body, which raises an issue of difficulty with accurate biopotential measurement. As a method of preventing such an issue, a method has been proposed that coats a contact portion to be brought into contact with the skin with silver chloride (AgCl); however, a portion of the silver chloride (AgCl) is likely to drop off, and thus improvement in the mechanical reliability is desired.

In contrast, in the present embodiment, the conductive material and the non-polarizable material having the non-polarizable property and ionic bonding are dispersed in the substrate that forms the electrode 1, and the regions different from each other in the concentration ratio between the conductive material and the non-polarizable material are formed in a contact portion to be brought into contact with an object and a non-contact portion of the electrode 1. Specifically, the first region 11 with higher concentration of the non-polarizable material than that of the conductive material is formed in the contact portion to be brought into contact with the object, and the second region 12 with higher concentration of the conductive material than that of the non-polarizable material is formed in the non-contact portion. In a case where the object is a living body, this makes it possible to prevent polarization of the contact portion brought into contact with the living body. Further, the non-polarizable material is dispersed in the substrate along with the conductive material, and the first region 11 having the non-polarizable material in high concentration and the second region 12 are formed in an integrated manner, which makes it possible to prevent drop-off of a portion of the non-polarizable material, or the like.

As described above, in the electrode 1 of the present embodiment, the conductive material and non-polarizable material are dispersed in the substrate; the first region 11 and the second region 12 are formed that are different from each other in a concentration ratio; and the first region 11 with higher concentration of the non-polarizable material than that of the conductive material is used as the contact portion to be brought into contact with a living body. This ensures to reduce polarization of the contact portion of the electrode 1 brought into contact with the living body, which allows for accurate biopotential measurement. Further, the first region 11 having the non-polarizable material in high concentration is formed in an integrated manner as the electrode 1, which prevents drop-off of a portion of the non-polarizable material, or the like. This makes it possible to provide an electrode that allows for the accurate biopotential measurement and exhibits the improved electrical and mechanical reliability.

Further, in the present embodiment, it is possible to distribute the non-polarizable material in high concentration at a desired local position through, for example, sedimentation in a fluidized state, centrifugal separation, or the like. This makes it possible to manufacture an electrode that exhibits the improved electrical and mechanical reliability at low cost and with ease.

### 2. APPLICATION EXAMPLES

Next, description is provided of application examples of an electronic apparatus that includes the electrode 1 (or any of the electrodes 1A to 1C) described in the above embodiment. However, the configuration of the electronic apparatus described below is merely an example, and it is possible to change the configuration as appropriate. The above-described electrode 1 is applicable to various sensors, various electronic apparatuses, or a portion of furnishings that detect or measure, for example, perspiration, body temperature, a perspiration ingredient, skin gas, blood sugar, and the like. For example, the above-described electrode 1 is applicable, as a so-called wearable device, to a portion of furnishings such as a watch (a wristwatch), a bag, clothes, a hat or a cap, glasses, and shoes. Types of the applicable electronic apparatuses and the like are not particularly limited.

### Application Example 1

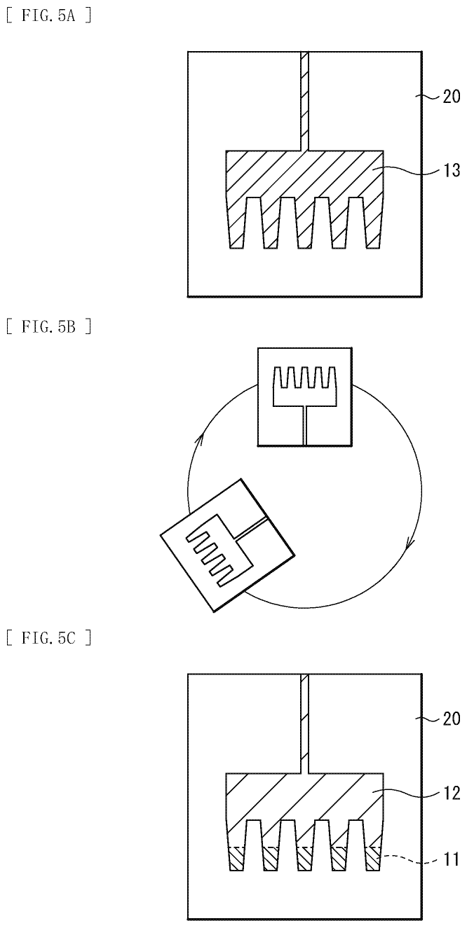

FIG. 6 illustrates a schematic configuration of a biopotential sensor. The electrode 1 of the present embodiment is usable as a measuring section (a sensor 110) that allows for measuring a biopotential or a bioimpedance by performing modification or geometric processing on an electrode surface on an as-needed basis, and by coupling the electrode 1 to a controller 120, a wiring line 130, and a circuit 140.

### Application Example 2

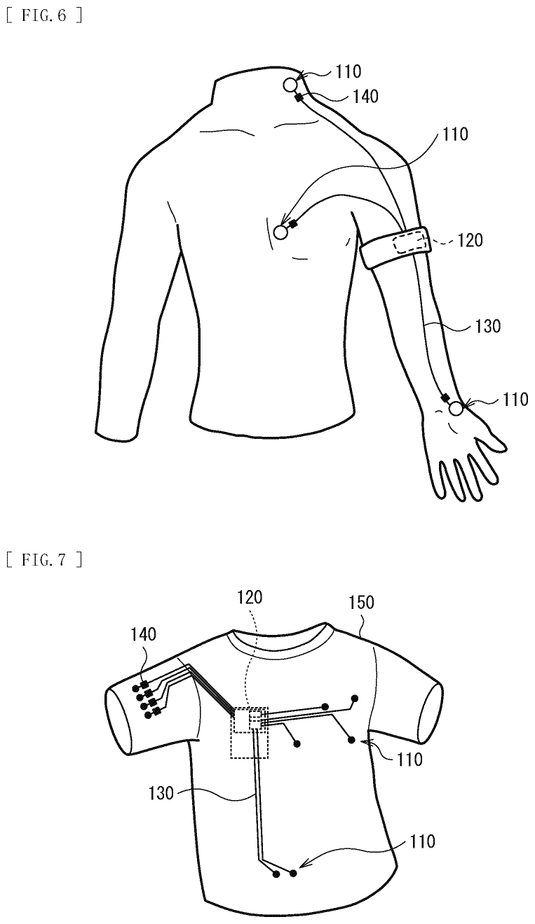

FIG. 7 illustrates an external appearance of clothing 150. The above-described application example 1 illustrates an example where the sensor 110 is mounted directly on a living body; however, the sensor 110 may be mounted on the clothing 150 or the like, as illustrated in FIG. 7, for example. The clothing 150 includes the sensor 110, the controller 120 that controls the sensor 110, the sensor 110, and the controller 120. It is to be noted that the circuit 140 may be provided in the middle of a path of the wiring line 130 between the sensor 110 and the controller 120.

The present disclosure is described thus far with reference to the embodiment and the application examples; however, the present disclosure is not limited to aspects described in the above embodiment and the like, but various modifications may be made. For example, it is not necessary to provide all of the component parts described in the above embodiment and the like, and as an alternative, any other component parts may be further included. Further, the material and the like for each of the above-described component parts are merely an example and not limited to those described above.

It is to be noted that the effects described herein are merely exemplified and non-limiting, and the effects of the present disclosure may be other effects, or may further include other effects.

It is to be noted that the present disclosure may be configured as follows.
(1) An electrode including:

a first conductive material;

a second conductive material having non-polarizable property and ionic bonding; and

a substrate that includes the first conductive material and the second conductive material, and has a first region and a second region that are different from each other in a concentration ratio between the first conductive material and the second conductive material.
(2) The electrode according to (1), in which the first region is a contact portion to be brought into contact with an object, and includes the second conductive material in a higher concentration than the second region.
(3) The electrode according to (1) or (2), further including, in the vicinity of an interface between the first region and the second region, a concentration gradient where a concentration ratio between the first conductive material and the second conductive material varies continuously.
(4) The electrode according to any one of (1) to (3), in which

the substrate includes a resin material, and

the first conductive material and the second conductive material are dispersed in the resin material.
(5) The electrode according to any one of (1) to (4), in which an average primary particle size of the first conductive material is smaller than an average primary particle size of the second conductive material.
(6) The electrode according to (4) or (5), in which a specific gravity of the first conductive material is smaller than a specific gravity of the second conductive material and a specific gravity of the resin material.
(⁷) The electrode according to any one of (1) to (6), in which the first conductive material has a polycarboxylic acid-based, urethane-based, or acrylic resin-based modifying group.
(8) The electrode according to (7), in which the modifying group is a triisostearoyl titanate-based coupling agent, a silane coupling agent, thiol, or phosphate ester.
(9) The electrode according to any one of (1) to (8), in which the first conductive material is a particle containing carbon as a main constituent.
(10) The electrode according to any one of (1) to (9), in which the first conductive material is a carbon particle, a carbon-based material particle, a metal particle, or a nanowire thereof.
(11) The electrode according to any one of (1) to (10), in which the second conductive material is a particle or a fiber of a metal compound, a metal oxide, or a conductive polymer.
(12) The electrode according to any one of (1) to (11), in which the second conductive material is silver chloride (AgCl), copper sulfide (CuS), palladium oxide (PdO2), indium tin oxide (ITO), PEDOT-PSS, PEDOT-TsO, or polyaniline.
(13) The electrode according to any one of (2) to (12), in which the object is a living body.
(14) A sensor having a measurement section that measures information on an object, the measurement section including an electrode, the electrode including:
a first conductive material;
a second conductive material having non-polarizable property and ionic bonding; and
a substrate that includes the first conductive material and the second conductive material, and has a first region and a second region that are different from each other in a concentration ratio between the first conductive material and the second conductive material.

This application claims the priority on the basis of Japanese Patent Application No. 2018-026342 filed on Feb. 16, 2018 with Japan Patent Office, the entire contents of which are incorporated in this application by reference.

It should be understood by those skilled in the art that various modifications, combinations, sub-combinations, and alterations may occur depending on design requirements and other factors insofar as they are within the scope of the appended claims or the equivalents thereof.

1. An electrode comprising:
a first conductive material;
a second conductive material having non-polarizable property and ionic bonding; and
a substrate that includes the first conductive material and the second conductive material, and has a first region and a second region that are different from each other in a concentration ratio between the first conductive material and the second conductive material.

2. The electrode according to claim 1, wherein the first region is a contact portion to be brought into contact with an object, and includes the second conductive material in a higher concentration than the second region.

3. The electrode according to claim 1, further comprising, in the vicinity of an interface between the first region and the second region, a concentration gradient where a concentration ratio between the first conductive material and the second conductive material varies continuously.

4. The electrode according to claim 1, wherein
the substrate includes a resin material, and
the first conductive material and the second conductive material are dispersed in the resin material.

5. The electrode according to claim 1, wherein an average primary particle size of the first conductive material is smaller than an average primary particle size of the second conductive material.

6. The electrode according to claim 4, wherein a specific gravity of the first conductive material is smaller than a specific gravity of the second conductive material and a specific gravity of the resin material.

7. The electrode according to claim 1, wherein the first conductive material has a polycarboxylic acid-based, urethane-based, or acrylic resin-based modifying group.

8. The electrode according to claim 7, wherein the modifying group comprises a triisostearoyl titanate-based coupling agent, a silane coupling agent, thiol, or phosphate ester.

9. The electrode according to claim 1, wherein the first conductive material comprises a particle containing carbon as a main constituent.

10. The electrode according to claim 1, wherein the first conductive material comprises a carbon particle, a carbon-based material particle, a metal particle, or a nanowire thereof.

11. The electrode according to claim 1, wherein the second conductive material comprises a particle or a fiber of a metal compound, a metal oxide, or a conductive polymer.

12. The electrode according to claim 1, wherein the second conductive material comprises silver chloride (AgCl), copper sulfide (CuS), palladium oxide (PdO₂), indium tin oxide (ITO), PEDOT-PSS, PEDOT-TsO, or polyaniline.

13. The electrode according to claim 2, wherein the object comprises a living body.

14. A sensor having a measurement section that measures information on an object, the measurement section including an electrode, the electrode comprising:
a first conductive material;
a second conductive material having non-polarizable property and ionic bonding; and
a substrate that includes the first conductive material and the second conductive material, and has a first region and a second region that are different from each other in a concentration ratio between the first conductive material and the second conductive material.
